(19) United States
(12) Patent Application Publication
(10) Pub. No.: US 2003/0194386 A1
(43) Pub. Date: Oct. 16, 2003

(54) Moisture-curable cosmetic composition

(75) Inventors: Pascale Bernard, Sucy-en-Brie (FR); Roland Ramin, Paris (FR); Jean Mondet, Aulnay-sous-Bois (FR)

Correspondence Address: STEPTOE & JOHNSON LLP, 1330 Connecticut Ave., N.W., Washington, DC, 20036, US

(73) Assignee: L' OREAL, Paris (FR)

(21) Appl. No.: 10/115,292

(22) Filed: Apr. 4, 2002

(51) Int. Cl. A61K 7/04

(52) U.S. Cl. 424/61

(57) ABSTRACT

The invention relates to the use, as a nail varnish, of a moisture-cure cosmetic composition comprising, in a cosmetically acceptable anhydrous medium, and free of compounds containing labile hydrogen atoms, i) either

a) at least one first compound, known as compound A, comprising at least two non-blocked reactive functions X, and

b) at least one second compound, known as compound B, comprising at least two blocked reactive functions Y, which may be unblocked by the action of moisture so as to be able to react with the non-blocked reactive functions X of the compound(s) A,

ii) or at least one compound simultaneously comprising at least two non-blocked reactive functions X and at least two blocked reactive functions Y, which may be unblocked by the action of moisture so as to be able to react with the non-blocked reactive functions X,

the average functionality of the system, that is to say the total number of non-blocked reactive functions X and of blocked reactive functions Y relative to the total number of molecules of compounds, being strictly greater than 2.

The present invention relates to moisture-cure cosmetic compositions, in particular nail varnishes, and also to a process for coating keratin materials using these compositions.

It is known from the field of paints that the crosslinking of a polymer deposit considerably improves its mechanical strength (friction strength and impact strength) and chemical resistance (resistance to solvents or oils).

Deposits on the nails are generally obtained by simple drying—involving no chemical reaction—of cosmetic compositions mainly containing pigments or colorants, film-forming polymers and volatile organic solvents.

These deposits do not always have a satisfactory staying power and have the drawback of needing to be renewed at regular intervals, which places an unwelcome constraint on the user.

Patent application NL-A-6 911 125 discloses pigmented coatings for the dental field or for nail varnishes, containing urethane prepolymers bearing free NCO groups which cure on application with atmospheric moisture. However, the crosslinking of these compositions is directly associated with the amount of moisture, to the extent that it is impossible to control the degree of crosslinking of the film obtained.

The Applicant has discovered that it is possible to obtain varnishes with good properties of chemical resistance and mechanical strength by performing the crosslinking, in particular of urethane prepolymers, with multifunctional compounds comprising reactive groups. These cosmetic compositions, and in particular nail varnishes, are capable of being crosslinked in situ and thus of forming films that have the abovementioned properties.

In this perspective, it appears to be difficult from a practical point of view to envisage the use of two reagents kept in separate containers and mixed together immediately before the application.

The storage, in the same container, of two reagents capable of reacting with each other at a precise moment thus poses the problem of the scorching of the system, which must be avoided at all costs.

The Applicant has solved this problem by blocking one of the two types of reactive function involved in the crosslinking reaction. The blocked functions are unblocked, after applying the cosmetic composition to the substrate, by the action of moisture, and the functions thus unblocked can then react with the non-blocked reactive functions of the compounds also present in the composition.

These moisture-cure cosmetic compositions show good stability over time, and allow the production of crosslinked films that show good resistance to water, to solvents such as fragrances or oils, to friction and to impacts, thus avoiding any wear or chipping.

One subject of the present invention is, consequently, a moisture-cure cosmetic composition comprising components containing reactive functions, of which some are blocked and others are not blocked, the blocked functions being able to be unblocked by moisture.

A subject of the invention is also a process for coating a keratin substrate.

Another subject consists of the use of the abovementioned compositions as nail varnishes.

Other objects of the invention will become apparent on reading the description and the examples which follow.

The composition that is the subject of the invention is a moisture-cure cosmetic composition comprising, in a cosmetically acceptable anhydrous medium, and free of compounds containing labile hydrogen atoms, i) either

a) at least one first compound, known as compound A, comprising at least two non-blocked reactive functions X, and

b) at least one second compound, known as compound B, comprising at least two blocked reactive functions Y, which may be unblocked by the action of moisture so as to be able to react with the non-blocked reactive functions X of the compound(s) A,

ii) or at least one compound simultaneously comprising at least two non-blocked reactive functions X, and at least two blocked reactive functions Y, which may be unblocked by the action of moisture so as to be able to react with the non-blocked reactive functions X.

In order for the reactive system formed by compounds A and B or by the compound(s) bearing the functions X and Y to be able to form a crosslinked macromolecular network, its average functionality, that is to say the total number of non-blocked reactive functions X and of blocked reactive functions Y relative to the total number of molecules of compounds, must be strictly greater than 2. The reason for this is that an average functionality of less than or equal to 2 would simply give a linear or branched polymer system.

In order to obtain a satisfactory crosslinking effect, the average functionality of the crosslinking system of the cosmetic compositions of the present invention is preferably at least equal to 2.2, especially ranging from 2.2 to 100 and better still ranging from 2.5 to 100.

In one embodiment of the cosmetic compositions of the present invention, a more or less large fraction or all of the compounds A and/or B can bear, respectively, in addition to the non-blocked reactive functions X and/or the blocked reactive functions Y intrinsic thereto, one or more “coreactive” functions. In other words, some or all of the compounds A can bear, in addition to the non-blocked reactive functions X, one or more blocked reactive functions Y and, similarly, some or all of the compounds B can bear, in addition to the blocked reactive functions Y, one or more non-blocked reactive functions X.

In one particular embodiment of the present invention, the combination of compounds forming the crosslinking system present in the composition comprises both non-blocked reactive functions X and blocked reactive functions Y.

According to one embodiment of the invention, the moisture bringing about the crosslinking can come from the air and/or from the support onto which the said composition is applied.

According to the present invention, the non-blocked reactive functions X are especially chosen from isocyanate and epoxide functions and ethylenic double bonds.

Compounds Containing Isocyanate Functions:

Compounds comprising at least two free isocyanate functions are known in the art. These may be polyisocyanates possibly having a low molecular mass of less than 1 000 000, including diisocyanates, triisocyanates or polyisocyanates which may have a molecular mass of less than 10 000. These polyisocyanates are generally obtained by polyaddition, polycondensation and/or grafting, bearing two or more than two isocyanate functions either at the chain ends or on the side groups. The polyisocyanates may be linear or branched, and aliphatic, cycloaliphatic or aromatic.

Examples of such compounds that may be mentioned include:

a) diisocyanates containing from 4 to 50 and preferably from 4 to 30 carbon atoms, such as 1,4-tetramethylene diisocyanate, 1,6-hexamethylene diisocyanate, 2,6- and 2,4-toluene diisocyanate, diphenylmethane diisocyanate and isophorone diisocyanate.

b) triisocyanates of formulae

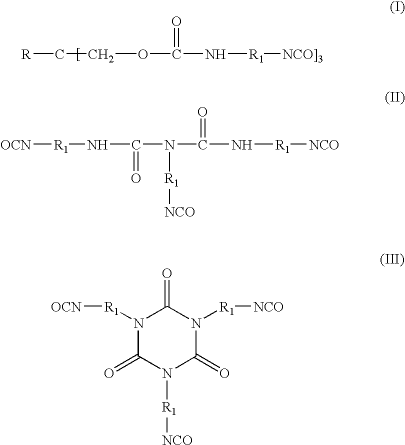

in which R is an alkyl radical containing from 1 to 30 carbon atoms, each R₁ independently represents a linear, branched or cyclic divalent hydrocarbon-based radical containing from 2 to 30 carbon atoms.

c) polycondensates containing isocyanate end groups or side groups, such as polyurethanes and/or polyureas (including blocked copolymers comprising at least one polyurethane and/or polyurea block and at least one polyether, polyester, polysiloxane, alkyd or polyacrylate block), and also polyesters, polyamides, polyepoxy, polyethers and perfluoropolyethers.

d) polymers resulting from the copolymerization of vinyl, allylic and/or (meth)acrylic monomers and of ethylenically unsaturated comonomers comprising a free isocyanate function, for instance 2-iso -cyanatoethyl methacrylate.

Polyisocyanates that may be used include Desmodur® N from the company Bayer and Tolonate® HDB-LV from the company Rhodia.

Compounds Containing an Epoxide Function:

Compounds comprising at least two epoxide functions are known in the prior art. They may be of any chemical nature. They may be diepoxides or polyepoxides of low mass (less than or equal to 5 000), or oligomers or polymers of any chemical nature, obtained by polyaddition, polycondensation and/or grafting, bearing at least two free epoxide functions, either at the chain ends or as side groups. Examples of such compounds that may be mentioned include:

a) bisphenol A diglycidyl ether resulting from the condensation between bisphenol A and epichlorohydrin, of structure

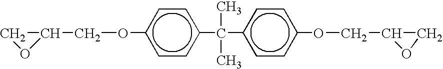

b) diepoxy resins resulting from the higher condensation between bisphenol A diglycidyl ether and epichlorohydrin,

c) epoxy ester resins containing α,ω-diepoxy end groups resulting from the condensation of a dicarboxylic acid especially containing from 2 to 60 carbon atoms with a stoichiometric excess of compounds a) or b),

d) epoxy ether resins containing α,ω-diepoxy end groups resulting from the condensation of a diol especially containing from 2 to 60 carbon atoms with a stoichiometric excess of compounds a) or b),

e) natural or synthetic oils bearing at least two epoxide groups, such as, for example, epoxidized soybean oil, epoxidized linseed oil or vernonia oil, especially described in patent application EP-A-645 134,

f) oligomers or polymers resulting from the copolymerization of unsaturated or vinyl, allylic and/or (meth)acrylic monomers, and of ethylenically unsaturated comonomers comprising a free epoxide function (for instance glycidyl methacrylate),

g) other polycondensates containing epoxy end groups and/or side groups, such as polyesters, polyesteramides, polyamides, alkyds, polyurethanes and/or polyureas, polyethers and perfluoropolyethers or silicones.

Polymers containing epoxy functions are sold under the names Cyracure® UVR-6110, Cyracure® UVR-6105, Cyracure® ERL-4221E, Cyracure® ERL-4206, Cyracure® UVR 6128 and Cyracure® UVR 6216 by the company Union Carbide, DER® 439 by the company Dow Chemical, Epikates® 828, 1001, 1004 and 1007 from the company Shell, Araldite® ECN1299 from the company Ciba-Geigy, and Epoxy Novolacs® from the company Dow Chemical.

Compounds Containing Ethylenic Double Bonds:

The compounds bearing ethylenic double bonds may be of any chemical nature. They may especially be chosen from:

a) ethylenically unsaturated polyesters:

This is a group of polymers of polyester type containing one or more ethylenic double bonds, randomly distributed in the main chain of the polymer. These unsaturated polyesters are obtained by polycondensation of a mixture

of linear or branched aliphatic or cycloaliphatic dicarboxylic acids especially containing from 3 to 50 carbon atoms and preferably from 3 to 20 carbon atoms, such as adipic acid or sebacic acid, of aromatic dicarboxylic acids especially containing from 8 to 50 carbon atoms and preferably from 8 to 20 carbon atoms, such as phthalic acids, especially terephthalic acid, and/or of dicarboxylic acids derived from ethylenically unsaturated fatty acid dimers such as the oleic acid or linoleic acid dimers described in patent application EP-A-959 066 (paragraph [0021]) sold under the names Pripol® by the company Unichema or Empol® by the company Henkel, all these diacids needing to be free of polymerizable ethylenic double bonds,

of linear or branched aliphatic or cycloaliphatic diols especially containing from 2 to 50 carbon atoms and preferably from 2 to 20 carbon atoms, such as ethylene glycol, diethylene glycol, propylene glycol, 1,4-butanediol or cyclohexane-dimethanol, of aromatic diols containing from 6 to 50 carbon atoms and preferably from 6 to 20 carbon atoms, such as bisphenol A or bisphenol B, and/or of diol diners derived from the reduction of the fatty acid diners as defined above, and

of one or more dicarboxylic acids or anhydrides thereof comprising at least one polymerizable ethylenic double bond and containing from 3 to 50 carbon atoms and preferably from 3 to 20 carbon atoms, such as maleic acid, fumaric acid or itaconic acid.

b) polyesters containing (meth)acrylate side groups and/or end groups:

This is a group of polymers of polyester type obtained by polycondensation of a mixture

of linear or branched aliphatic or cycloaliphatic dicarboxylic acids especially containing from 3 to 50 carbon atoms and preferably from 3 to 20 carbon atoms, such as adipic acid or sebacic acid, of aromatic dicarboxylic acids especially containing from 8 to 50 carbon atoms and preferably from 8 to 20 carbon atoms, such as phthalic acids, especially terephthalic acid, and/or of dicarboxylic acids derived from ethylenically unsaturated fatty acid dimers such as the oleic acid or linoleic acid dimers described in patent application EP-A-959 066 (paragraph [0021]) sold under the names Pripol® by the company Unichema or Empol® by the company Henkel, all these diacids needing to be free of polymerizable ethylenic double bonds,

of linear or branched aliphatic or cycloaliphatic diols especially containing from 2 to 50 carbon atoms and preferably from 2 to 20 carbon atoms, such as ethylene glycol, diethylene glycol, propylene glycol, 1,4-butanediol or cyclohexane-dimethanol, of aromatic diols containing from 6 to 50 carbon atoms and preferably from 6 to 20 carbon atoms, such as bisphenol A or bisphenol B, and

of at least one monoester of (meth)acrylic acid and of a diol or polyol containing from 2 to 20 carbon atoms and preferably from 2 to 6 carbon atoms, such as 2-hydroxyethyl (meth)acrylate, 2-hydroxypropyl (meth)acrylate or glyceryl methacrylate.

These polyesters differ from those described above in point a) by the fact that the ethylenic double bonds are not located in the main chain but on side groups or at the end of the chains. These ethylenic double bonds are those of the (meth)acrylate groups present in the polymer.

Such polyesters are sold, for example, by the company UCB under the names Ebecryl® (Ebecryl® 450: molar mass 1 600, on average 6 acrylate functions per molecule, Ebecryl® 652: molar mass 1 500, on average 6 acrylate functions per molecule, Ebecryl® 800: molar mass 780, on average 4 acrylate functions per molecule, Ebecryl® 810: molar mass 1 000, on average 4 acrylate functions per molecule, Ebecryl® 50 000: molar mass 1 500, on average 6 acrylate functions per molecule).

c) polyurethanes and/or polyureas containing (meth)acrylate groups, obtained by polycondensation

of aliphatic, cycloaliphatic and/or aromatic diisocyanates, triisocyanates and/or polyisocyanates especially containing from 4 to 50 and preferably from 4 to 30 carbon atoms, such as hexamethylene diisocyanate, isophorone diisocyanate, toluene diisocyanate, diphenylmethane diisocyanate or isocyanurates of formula

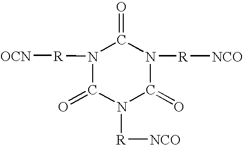

resulting from the trimerization of 3 diisocyanate molecules OCN—R—CNO, in which R is a linear, branched or cyclic hydrocarbon-based radical containing from 2 to 30 carbon atoms;

of polyols, especially of diols, free of polymerizable ethylenic unsaturation, such as 1,4-butanediol, ethylene glycol or trimethylolpropane, and/or of aliphatic, cycloaliphatic and/or aromatic polyamines, especially diamines, especially containing from 3 to 50 carbon atoms, such as ethylenediamine or hexamethylenediamine, and

of at least one monoester of (meth)acrylic acid and of a diol or polyol containing from 2 to 20 carbon atoms and preferably from 2 to 6 carbon atoms, such as 2-hydroxyethyl (meth)acrylate, 2-hydroxypropyl (meth)acrylate and glyceryl methacrylate.

Such polyurethanes/polyureas containing acrylate groups are sold, for example, under the name SR 368 (tris(2-hydroxyethyl) isocyanurate-triacrylate) or Craynor® 435 by the company Cray Valley, or under the name Ebecryl® by the company UCB (Ebecryl® 210: molecular mass 1 500, 2 acrylate functions per molecule, Ebecryl® 230: molecular mass 5 000, 2 acrylate functions per molecule, Ebecryl® 270: molecular mass 1 500, 2 acrylate functions per molecule, Ebecryl® 8402: molecular mass 1 000, 2 acrylate functions per molecule, Ebecryl® 8804: molecular mass 1 300, 2 acrylate functions per molecule, Ebecryl® 220: molecular mass 1 000, 6 acrylate functions per molecule, Ebecryl® 2220: molecular mass 1 200, 6 acrylate functions per molecule, Ebecryl® 1290: molecular mass 1 000, 6 acrylate functions per molecule, Ebecryl® 800: molecular mass 800, 6 acrylate functions per molecule). Mention may also be made of the water-soluble aliphatic diacrylate polyurethanes sold under the names Ebecryl® 2000, Ebecryl® 2001 and Ebecryl® 2002, and the diacrylate polyurethanes in aqueous dispersion sold under the trade names IRR® 390, IRR® 400, IRR® 422 and IRR® 424 by the company UCB.

d) polyethers containing (meth)acrylate groups obtained by esterification, with (meth)acrylic acid, of the hydroxyl end groups of C₁₋₄ alkylene glycol homopolymers or copolymers, such as polyethylene glycol, polypropylene glycol, copolymers of ethylene oxide and of propylene oxide preferably having a weight-average molecular mass of less than 10 000, and polyethoxylated or polypropoxylated trimethylolpropane.

Polyoxyethylene di(meth)acrylates of suitable molar mass are sold, for example, under the names SR 259, SR 344, SR 610, SR 210, SR 603 and SR 252 by the company Cray Valley or under the name Ebecryl® 11 by UCB. Polyethoxylated trimethylolpropane triacrylates are sold, for example, under the names SR 454, SR 498, SR 502, SR 9035 and SR 415 by the company Cray Valley or under the name Ebecryl® 160 by the company UCB. Polypropoxylated trimethylolpropane triacrylates are sold, for example, under the names SR 492 and SR 501 by the company Cray Valley.

e) epoxyacrylates obtained by reaction between

at least one diepoxide chosen, for example, from:

1) bisphenol A diglycidyl ether,

2) a diepoxy resin resulting from the reaction between bisphenol A diglycidyl ether and epichlorohydrin,

3) an epoxy ester resin containing α,ω-diepoxy end groups resulting from the condensation of a dicarboxylic acid containing from 3 to 50 carbon atoms with a stoichiometric excess of 1) and/or 2), and

4) an epoxy ether resin containing α,ω-diepoxy end groups resulting from the condensation of a diol containing from 3 to 50 carbon atoms with a stoichiometric excess of 1) and/or 2),

5) natural or synthetic oils bearing at least 2 epoxide groups, such as epoxidized soybean oil, epoxidized linseed oil or epoxidized vernonia oil,

6) a phenol-formaldehyde polycondensate (Novolac® resin), the end groups and/or side groups of which have been epoxidized, and

one or more carboxylic acids or polycarboxylic acids comprising at least one ethylenic double bond in the α,β-position relative to the carboxylic group, for instance (meth)acrylic acid or crotonic acid or monoesters of (meth)acrylic acid and of a diol or polyol containing from 2 to 20 carbon atoms and preferably from 2 to 6 carbon atoms, such as 2-hydroxyethyl (meth)acrylate.

Such polymers are sold, for example, under the names SR 349, SR 601, CD 541, SR 602, SR 9036, SR 348, CD 540, SR 480 and CD 9038 by the company Cray Valley, under the names Ebecryl® 600, Ebecryl® 609, Ebecryl® 150, Ebecryl® 860 and Ebecryl® 3702 by the company UCB and under the names Photomer® 3005 and Photomer® 3082 by the company Henkel.

f) poly(C₁₋₅₀ alkyl (meth)acrylates) comprising at least two functions containing an ethylenic double bond borne by the hydrocarbon-based side chains and/or end chains.

Such copolymers are sold, for example, under the names IRR® 375, OTA® 480 and Ebecryl® 2047 by the company UCB.

g) polyorganosiloxanes containing (meth)acrylate or (meth)acrylamide groups obtained, respectively,

by esterification, for example with (meth)acrylic acid, of polyorganosiloxanes, preferably of polydimethylsiloxanes (PDMSs), bearing hydroxyl end groups and/or side groups,

by amidation, for example with (meth)acrylic acid, of polyorganosiloxanes bearing primary or secondary amine side groups and/or end groups.

Hydroxylated PDMSs that may be mentioned in particular are PDMSs comprising at least two C₁₋₆ hydroxyalkyl groups and dimethicone copolyols with hydroxyl side groups or end groups.

Esterifiable α,ω-dihydroxylated polydimethylsiloxanes are sold under the names Tegomer® H-Si 2111 and Tegomer® H-Si 2311 by the company Goldschmidt. α,ω-Diacrylate polydimethylsiloxanes are available from the company Shin-Etsu under the references X-22-164 B and X-22-164 C.

Amino PDMSs that may be mentioned in particular are PDMSs comprising at least 2 C₁₋₁₀ aminoalkyl groups, for example the aminosilicone sold under the name Q2-8220 by the company Dow Corning.

Advantageously, the silicone polymers of this group are used as a mixture with one or more polymers of other groups a) to f) described above, especially to modify the hydrophobic nature of the final composition.

h) perfluoropolyethers containing acrylate groups obtained by esterification, for example with (meth)acrylic acid, of perfluoropolyethers bearing hydroxyl side groups and/or end groups.

Such α,ω-diol perfluoropolyethers are described especially in EP-A-1 057 849 and are sold by the company Ausimont under the name Fomblin® Z Diol.

i) hyperbranched dendrimers and polymers bearing (meth)acrylate or (meth)acrylamide end groups obtained, respectively, by esterification or amidation of hyperbranched dendrimers and polymers containing hydroxyl or amino end functions, with (meth)acrylic acid.

Dendrimers (from the Greek dendron=tree) are “arborescent”, that is to say highly branched, polymer molecules invented by D. A. Tomalia and his team at the start of the 1990s (Donald A. Tomalia et al., Angewandte Chemie, Int. Engl. Ed., Vol. 29, No. 2, pages 138-175). These are structures constructed about a central unit that is generally polyvalent. About this central unit are linked, in a fully determined structure, branched chain-extending units, thus giving rise to monodispersed symmetrical macromolecules having a well-defined chemical and stereochemical structure. Dendrimers of polyamidoamine type are sold, for example, under the name Starburst® by the company Dendritech.

Hyperbranched polymers are polycondensates, generally of polyester, polyamide or polyethyleneamine type, obtained from multifunctional monomers, which have an arborescent structure similar to that of dendrimers but are much less regular than dendrimers (see, for example, WO-A-93/17060 and WO 96/12754).

The company Perstorp sells hyperbranched polyesters under the name Boltorn®. Hyperbranched polyethyleneamines will be found under the name Comburst® from the company Dendritech. Hyperbranched poly(esteramides) containing hydroxyl end groups are sold by the company DSM under the name Hybrane®.

These hyperbranched dendrimers and polymers esterified or amidated with acrylic acid and/or methacrylic acid are distinguished from the polymers described in points a) to h) above by the very large number of ethylenic double bonds present. This high functionality, usually greater than 5, makes them particularly useful by allowing them to act as “crosslinking nodes”, that is to say sites of multiple crosslinking.

In one preferred embodiment of the invention, these dendritic and hyperbranched polymers will consequently be used in combination with one or more of the polymers and/or oligomers a) to h) above.

The blocked reactive functions Y are chosen especially from amine functions blocked in ketimine and aldimine form and amino alcohol functions blocked in oxazolidine form.

The compounds bearing the blocked amine functions are chosen especially:

a) from the blocked polyamines of general formula

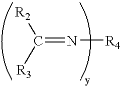

which are imines resulting from the reaction between a compound containing amine groups of formula R₄—(NH₂)_{y} and a ketone of formula

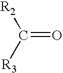

y being greater than or equal to 2, y preferably ranging from 2 to 100, R₂, which is identical to or different from R₃, being an alkyl group containing from one to four carbon atoms. Preferably, R₂=R₃=—CH₃, or R₂=—CH₃ and R₃=—C₂H₅, or R₂=—CH₃ and R₃=isobutyl or isopropyl.

The compound R₄—(NH₂)_{y} may be a diamine, a polyamine, an oligomer or a polymer containing amine groups, preferably chosen from

1. aliphatic, cycloaliphatic or aromatic diamines especially containing from 2 to 6 carbon atoms, such as ethylenediamine, 1,2-diaminopropane, 1,3-diaminopropane, 1,4-diaminobutane, 1,2-diamino-2-methylpropane, 1,6-diaminohexane, 1,10-di -aminodecane, isophoronediamine, adamantanediamine, 2,6-diaminopyridine or diamines obtained by modifying the ends of dimeric fatty acids,

2) multifunctional amines containing more than two amine groups, such as melanine, 2,4,6-triaminopyrimidine, 3,3′-diaminobenzidine or 2,4,5,6-tetraaminopyrimidine,

3) oligomers bearing at least two amine groups, such as the polyalkylene oxide diamines Jeffamine® from Texaco (polyetherdiamines),

4) oligomers or polymers (homopolymers and copolymers) bearing amine groups, preferably primary amine groups, located at the ends of the chains and/or on side chains, in particular vinylamine or allylamine homopolymers and copolymers, polycondensates of any nature bearing amine groups and in particular polyamides obtained by condensation of an excess of diamine,

5) hyperbranched dendrimers or polymers whose chain ends are primary amines, in particular polyamido amines such as those sold under the name Starburst® by the company Dendritech. The hyperbranched polymers are polycondensates generally of polyethyleneamine type obtained from multifunctional monomers, which have an arborescent structure similar to that of dendrimers but are much less regular than dendrimers.

Compounds containing blocked amine functions in ketimine form are sold especially under the name Epikure® H3 and Epikure 3505 by the company Shell, and Vestamin® A139 by the company Creanova.

b) from other compounds bearing blocked amine functions, chosen especially from the blocked polyamines of general formula

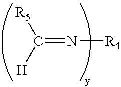

which are imines resulting from the reaction between a compound containing amine groups of formula R₄—(NH₂)_{y} and an aldehyde of formula

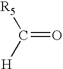

y being an integer greater than or equal to two, R₅ being an alkyl group containing from one to four carbon atoms, R₄ having the same meaning as above.

c) from other compounds, bearing amino alcohol functions blocked in oxazolidine form and chosen especially from the compounds of general formula

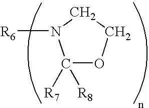

in which R₆ is a linear, branched, cyclic or heterocyclic hydrocarbon-based group containing from one to fifty carbon atoms, which can comprise one or more unsaturations, and which can comprise one or more hetero atoms such as O (which may especially be in the form of polyalkylene oxide groups), S, N, P or Si, the group R₆ possibly comprising reactive functions other than amine functions, which do not react with the non-blocked reactive functions, R₆ also possibly being a polymer or a dendrimer or a hyperbranched polymer, R₇ and R₈, which may be identical or different, are a hydrogen atom or an alkyl group containing from one to four carbon atoms, preferably with R₇=R₈=CH₃ or R₇=CH₃ and R8=C₂H₅, isopropyl or isobutyl,

n being a positive integer greater than or equal to two.

These compounds in oxazolidine form, in particular those for which n=2, are especially obtained by condensation between a compound comprising one or more amino alcohol functions of formula R₆—(NH—CH₂—CH₂—OH)ₙ and a ketone of formula

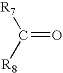

or an aldehyde of formula

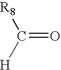

These compounds may also be obtained starting with a diamino alcohol, which is then generally diethanolamine, and by cyclizing in two stages:

cyclization of an amino alcohol group in the presence of a ketone (or an aldehyde), the second alcohol group not being affected by the reaction

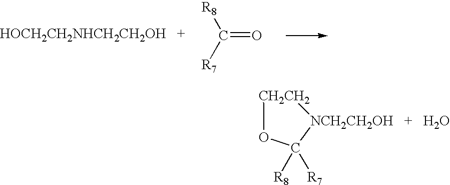

followed by reaction with a coupling agent containing at least two groups Y₁ possibly reacting with the hydroxyl under anhydrous conditions, not allowing opening of the oxazolidine ring formed according to the reaction

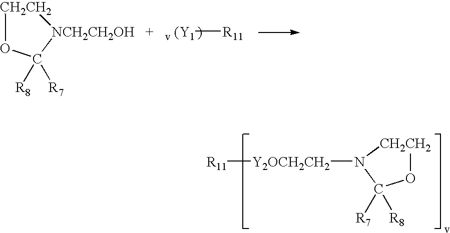

R₁₁ being equivalent to R₄ defined above, Y₂ being the group formed by the reaction between Y₁ and —OH.

In the above reaction, the compound of formula

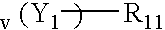

may be:

an aliphatic, cycloaliphatic or aromatic diisocyanate,

a diisocyanate trimer,

a triisocyanate of formula (II),

a polyisocyanate resulting from the reaction between an excess of diisocyanate and a polyol.

In each of these cases, Y₁=—NCO and Y₂ is represented by the formula

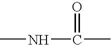

Similarly, the compound of formula

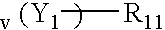

may be a diacid, triacid or polyacid or ester or acid chloride, the reaction taking place under anhydrous conditions. In this case, Y₁=—COOH or —COC1 and Y₂ is represented by the formula

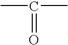

Similarly, the compound of formula

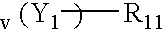

may be a diepoxide, triepoxide or polyepoxide, the reaction taking place under anhydrous conditions. In this case, Y₁ is represented by the formula

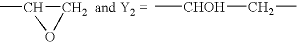

According to one particular embodiment of the invention, the composition may comprise at least one compound simultaneously comprising non-blocked reactive functions X and blocked reactive functions Y. Examples which may be mentioned include ketimines and aldimines blocking only two hydroxyl groups of a polyol such as glycerol or trimethylolpropane, the remaining hydroxyl group(s) then reacting with an excess of a diisocyanate, thus giving a compound bearing both ketimine (or aldimine) groups and several free isocyanate groups according to:

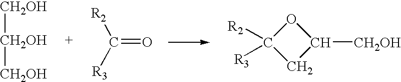

followed by

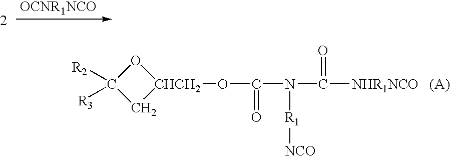

Compound A formed contains both a moisture-unblockable ketimine or aldimine group, and an allophanate reactive group (that is to say a group bearing two isocyanate reactive functions).

In this particular case, compound A may, when applied to a keratin support and in the presence of moisture, unblock the ketimine or aldimine group and coreact with itself giving a simultaneous polymerization and crosslinking reaction according to:

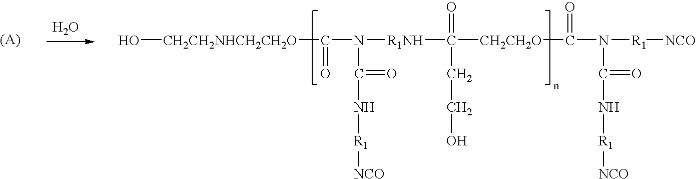

The —NCO and —OH functions bring about the cocrosslinking with another polymer chain formed and/or with the compound itself, to give a crosslinked network. These self-reactive compounds A and their reactions are especially mentioned by H. Renz, XXVIth International Conference in Organic Coatings, Athens, Jul. 2000, pp. 237-249, 2000.

Compounds containing amino alcohol functions blocked in oxazolidine form are especially described in documents WO-A-99/07763, JP-A-09-241501, WO-A-96/20231, WO-A-95/14528, U.S. Pat. No. 5 126 421, U.S. Pat. No. 4 381 388 and U.S. Pat. No. 4 504 647. They are sold under the names Incozol® 4 and Incozol® LV by the company Industrial Copolymer Ltd., Hardener® OZ by the company Bayer and Zoldine® RD-4 by the company Angus Chemicals Co.

The amine or hydroxyl functions corresponding to the ketimine, aldimine or oxazolidine functions are moisture-unblocked according to the following reactions:

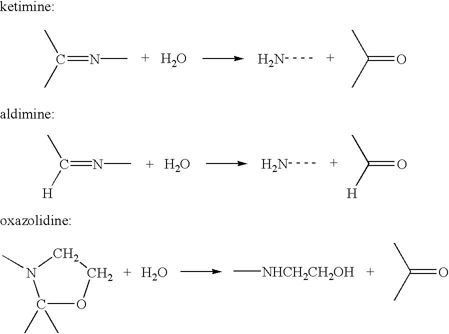

Once unblocked, the functions react in the usual manner with the N═C═O functions.

The moisture-cure cosmetic compositions of the present invention thus preferably contain one or more catalysts capable of accelerating the moisture-unblocking reaction of the blocked isocyanate functions. These catalysts are especially chosen from linear, branched or cyclic tertiary amines such as diazabicyclo[2.2.2]-octane, quinuclidine and 3,3,6,9,9-pentamethyl-2,10-diazabicyclo[4.4.0]dec-1-ene.

According to the present invention, the catalyst concentration is preferably between 0.1% and 2% by weight and preferably between 0.2% and 1% by weight, relative to the reagents.

The compounds A and B, or the compound(s) comprising both at least two non-blocked reactive functions and at least two blocked reactive functions, may be present in the composition according to the invention in a content ranging from 1% to 50% by weight and preferably ranging from 2% to 40% by weight, relative to the total weight of the composition. Preferably, the ratio of the total number of blocked reactive functions Y to the total number of non-blocked reactive functions X is greater than 1 and is especially up to 1.5.

The moisture-cure cosmetic compositions of the present invention may also contain one or more organic solvents. These solvents are chosen especially from physiologically acceptable organic solvents, among which mention may be made of

a) ketones that are liquid at room temperature, such as methyl ethyl ketone, methyl isobutyl ketone, diisobutyl ketone, isophorone, cyclohexanone and acetone,

b) alcohols that are liquid at room temperature, such as ethanol, isopropanol, diacetone alcohol, 2-butoxyethanol or cyclohexanol,

c) glycols that are liquid at room temperature, such as ethylene glycol, propylene glycol, pentylene glycol and glycerol,

d) propylene glycol ethers that are liquid at room temperature, such as propylene glycol monomethyl ether, propylene glycol monomethyl ether acetate and dipropylene glycol mono-n-butyl ether,

e) short-chain esters (containing in total from 3 to 8 carbon atoms), such as ethyl acetate, methyl acetate, propyl acetate, n-butyl acetate and isopentyl acetate,

f) alkanes that are liquid at room temperature, such as decane, heptane, dodecane and cyclohexane,

g) aromatic hydrocarbons that are liquid at room temperature, such as toluene and xylene,

h) silicones that are liquid at room temperature, and

i) mixtures thereof.

The solvent content in the composition may range from 0% to 80% by weight relative to the total weight of the composition and preferably from 1% to 60% by weight.

According to one particular embodiment of the composition according to the invention, the composition is free of solvent.

The reagents may be dissolved at the start in any type of volatile organic solvent that comprises no labile hydrogen atoms and that is free of water.

Thus, for better storage of the formula with respect to traces of water, according to one embodiment of the invention, a moisture absorber may be incorporated therein, which may be of any chemical nature, preferably solid in the form of microparticles, for instance silica, talc, kaolin, alumina silicates or diatomaceous earths.

The additives are chosen such that they do not react with the non-blocked reactive functions X and the blocked reactive functions Y of the moisture-cure compounds.

The abovementioned compositions may be used as nail varnishes.

A subject of the invention is also a process for coating keratin materials. This process comprises the application to a keratin substrate of a layer of a moisture-cure cosmetic composition described above. By the action of moisture from the air and/or from the support, the blocked reactive functions Y become unblocked and react with the non-blocked functions X, allowing the production of a partially or totally crosslinked film on the substrate.

The keratin substrates that can receive heat-crosslinkable coatings according to the present invention are, in particular, the nails, the eyelashes, the eyebrows and the hair, or makeup accessories such as false nails, false eyelashes or wigs.

The invention is described in greater detail by the examples below.

### EXAMPLES

### Example 1:

An atmospheric moisture-cure nail varnish having the composition below was prepared:

| | | |
|---|---|---|
| polymer containing an epoxide function | 21.6 | g |
| (Epikote ® 828 from Shell) | | |
| compound containing amine functions blocked | 8.4 | g |
| in ketimine form (Epikure ® H3 from Shell) | | |
| fumed silica (Aerosil ® 200 from Degussa) | 2.0 | g |
| dehydrated pigment | 3.0 | g |
| dehydrated toluene | 40 | g |
| dehydrated methyl isobutyl ketone | qs 100 g | |

The composition is applied to the nails in the form of a film which crosslinks on contact with moisture from the air. After drying, the dry film formed has good properties in terms of adhesion, gloss and staying power over time.

### Example 2:

An atmospheric moisture-cure nail varnish having the composition below was prepared:

| | | |
|---|---|---|
| polymer containing an epoxide function | 21 | g |
| (Epikote 215 from Shell) | | |
| compound containing amine functions blocked | 9 | g |
| in ketimine form (Epikure ® 3505 from Shell) | | |
| silica (Syloid ® 72 from Grace) | 2 | g |
| diamond powder | 0.001 | g |
| ceramide | 0.001 | g |
| dehydrated ethanol | 5 | g |
| dehydrated heptane | 30 | g |
| dehydrated methyl ethyl ketone | qs 100 g | |

The composition is applied to the nails in the form of a film which crosslinks on contact with moisture from the air. After drying, the dry film formed has good properties in terms of adhesion, gloss and staying power over time.

1. Use, as a nail varnish, of a moisture-cure cosmetic composition comprising, in a cosmetically acceptable anhydrous medium, and free of compounds containing labile hydrogen atoms, i) either
a) at least one first compound, known as compound A, comprising at least two non-blocked reactive functions X, and
b) at least one second compound, known as compound B, comprising at least two blocked reactive functions Y, which may be unblocked by the action of moisture so as to be able to react with the non-blocked reactive functions X of the compound(s) A,
ii) or at least one compound simultaneously comprising at least two non-blocked reactive functions X and at least two blocked reactive functions Y, which may be unblocked by the action of moisture so as to be able to react with the non-blocked reactive functions X,
the average functionality of the system, that is to say the total number of non-blocked reactive functions X and of blocked reactive functions Y relative to the total number of molecules of compounds, being strictly greater than 2.

2. Use according to claim 1, characterized in that some or all of the compounds A also bear one or more blocked reactive functions Y and/or some or all of the compounds B also bear one or more non-blocked reactive functions X.

3. Use according to claim 1 or 2, characterized in that the combination of compounds A and B comprises both non-blocked reactive functions X and blocked reactive functions Y.

4. Use according to any one of the preceding claims, characterized in that the moisture comes from the air and/or from the support onto which the said composition is applied.

5. Use according to any one of the preceding claims, characterized in that the non-blocked reactive functions X are chosen from isocyanate and epoxide functions and ethylenic double bonds.

6. Use according to any one of the preceding claims, characterized in that the blocked reactive functions Y are chosen from amine functions blocked in ketinime and aldimine form and amino alcohol functions blocked in oxazolidine form.

7. Use according to any one of the preceding claims, characterized in that it also comprises one or more organic solvents.

8. Use according to any one of the preceding claims, characterized in that it also comprises catalysts in proportions of between 0.1% and 2% by weight and preferably between 0.2% and 1% by weight, relative to the reagents.

9. Use according to any one of the preceding claims, characterized in that the compounds A and B or the compound(s) bearing both at least two non-blocked reactive functions and two blocked reactive functions are present in the composition in a content ranging from 1% to 50% by weight and preferably ranging from 2% to 40% by weight, relative to the total weight of the composition.

10. Use according to any one of the preceding claims, characterized in that it also comprises a moisture absorber.

11. Moisture-cure cosmetic composition comprising, in a cosmetically acceptable anhydrous medium, and free of compounds containing labile hydrogen atoms, i) either
a) at least one first compound, known as compound A, comprising at least two non-blocked reactive functions X, and
b) at least one second compound, known as compound B, comprising at least two blocked reactive functions Y, which may be unblocked by the action of moisture so as to be able to react with the non-blocked reactive functions X of the compound(s) A, the blocked reactive functions Y being chosen from the amine functions blocked in aldimine form and the amino alcohol functions blocked in oxazolidine form,
ii) or at least one compound simultaneously comprising at least two non-blocked reactive functions X and at least two blocked reactive functions Y, which may be unblocked by the action of moisture so as to be able to react with the non-blocked reactive functions X,
the average functionality of the system, that is to say the total number of non-blocked reactive functions x and of blocked reactive functions Y relative to the total number of molecules of compounds, being strictly greater than 2.

12. Moisture-cure cosmetic composition according to claim 11, characterized in that some or all of the compounds A also bear one or more blocked reactive functions Y and/or in that some or all of the compounds B also bear one or more non-blocked reactive functions X.

13. Moisture-cure cosmetic composition according to claim 11 or 12, characterized in that the combination of compounds A and B comprises both non-blocked reactive functions X and blocked reactive functions Y.

14. Moisture-cure cosmetic composition according to any one of claims 11 to 13, characterized in that the moisture comes from the air and/or from the support onto which the said composition is applied.

15. Moisture-cure cosmetic composition according to any one of claims 11 to 14, characterized in that the non-blocked reactive functions X are chosen from isocyanate and epoxide functions and ethylenic double bonds.

16. Moisture-cure cosmetic composition according to any one of claims 11 to 15, characterized in that the blocked reactive functions Y of the compound simultaneously comprising at least two non-blocked reactive functions X and at least two blocked reactive functions are chosen from amine functions blocked in ketinime and aldimine form and amino alcohol functions blocked in oxazolidine form.

17. Moisture-cure cosmetic composition according to any one of claims 11 to 16, characterized in that it also comprises one or more organic solvents.

18. Moisture-cure cosmetic composition according to any one of claims 11 to 17, characterized in that it also comprises catalysts in proportions of between 0.1% and 2% by weight and preferably between 0.2% and 1% by weight, relative to the reagents.

19. Moisture-cure cosmetic composition according to any one of claims 11 to 18, characterized in that the compounds A and B or the compound(s) bearing both at least two non-blocked reactive functions and two blocked reactive functions are present in the composition in a content ranging from 1% to 50% by weight and preferably ranging from 2% to 40% by weight, relative to the total weight of the composition.

20. Moisture-cure cosmetic composition according to any one of claims 11 to 19, characterized in that it also comprises a moisture absorber.

21. Process for coating a keratin substrate, characterized in that a composition defined in any one of claims 1 to 10 is applied to the said substrate.
